(19) United States
(12) Patent Application Publication
(10) Pub. No.: US 2004/0076674 A1
(43) Pub. Date: Apr. 22, 2004

(54) Method for thermally assisted antimicrobial surface treatment

(76) Inventors: Peter Ottersback, Windeck (DE); Beate Kossmann, Hagen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/432,630

(22) PCT Filed: Nov. 14, 2001
(86) PCT No.: PCT/EP01/13151

(30) Foreign Application Priority Data
Dec. 9, 2000 (DE) 100 61 250.4

(51) Int. Cl. B05D 3/00; A61K 9/14

(52) U.S. Cl. 424/486; 427/2.24

(57) ABSTRACT

The invention relates to a method for the antimicrobial treatment of surfaces by applying thermally assisted antimicrobial active polymers onto said surfaces.

The invention relates to a process for rendering surfaces antimicrobial through the thermally assisted application of antimicrobial polymers.

It is highly undesirable for bacteria to become established or to spread on the surfaces of pipelines, or of containers or packaging. Slime layers frequently form and permit sharp rises in microbial populations, and these can lead to persistent impairment of the quality of water or of drinks or foods, and even to spoilage of the product, and harm to the health of consumers.

Bacteria must be kept away from all fields of life where hygiene is important, this affects textiles for direct body contact, especially in the genital area, and those used for the care of the elderly or sick. Bacteria must also be kept away from the surface of the furniture and instruments used in patient-care areas, especially in areas for intensive care or neonatal care, and in hospitals, especially in areas where medical intervention takes place, and also in isolation wards for critical cases of infection, and in toilets.

A current method for treating equipment, or the surfaces of furniture or of textiles, to resist bacteria either when this becomes necessary or else as a precautionary measure is to use chemicals or solutions of these, or else mixtures, these being disinfectant and having fairly broad general antimicrobial action. Chemical agents of this type act nonspecifically and are themselves frequently toxic or irritant, or form degradation products which are hazardous to health. In addition, people frequently exhibit intolerance to these materials once they have become sensitized.

Another procedure for counteracting surface spread of bacteria is the incorporation of antimicrobial substances into a matrix.

Another challenge of constantly increasing significance is the avoidance of algal growth on surfaces, since there are many external surfaces of buildings with plastic cladding, which is particularly susceptible to colonization by algae. As well as giving an undesirable appearance, this can in some circumstances also impair the functioning of the components concerned. One relevant example is colonization by algae of surfaces with a photovoltaic function.

Another form of microbial pollution for which again no technically satisfactory solution has been found is fungal infestation of surfaces. For example, Aspergillus niger infestation of joints or walls in wet areas within buildings not only impairs appearance but also has serious health implications, since many people are allergic to the substances given off by the fungi, and the results can even be serious chronic respiratory disease.

In the marine sector, the fouling of boats' hulls affects costs, since the growth of fouling organisms is attended by an increase in the boat's flow resistance, and thus by a marked increase in fuel consumption. Problems of this type have hitherto generally been countered by incorporating toxic heavy metals or other low-molecular-weight biocides into antifouling coatings with the aim of mitigating the problems described. To this end, the damaging side effects of coatings of this type are accepted, but as society's environmental awareness rises this state of affairs is increasingly problematic.

U.S. Pat. No. 4,532,269, for example, therefore discloses a terpolymer made from butyl methacrylate, tributyltin methacrylate, and tert-butylaminoethyl methacrylate. This copolymer is used as an antimicrobial paint for ships, and the hydrophilic tert-butylaminoethyl methacrylate promotes slow erosion of the polymer, thus releasing. highly toxic tributyltin methacrylate as active antimicrobial agent.

In these applications, the copolymer prepared with aminomethacrylates is merely a matrix or carrier for added microbicidal active ingredients which can diffuse or migrate out of the carrier material. At some stage polymers of this type lose their activity once the necessary minimum inhibitor concentration (MIC) at the surface has been lost.

It is also known from European Patent Applications 0 862 858 that copolymers of tert-butylaminoethyl methacrylate, a methacrylate having a secondary amino function, have inherent microbicidal properties. For effective avoidance of undesirable resistance phenomena in microbes, particularly bearing in mind that the development of resistance by microbes is known from antibiotics research, systems developed in the future will again have to be based on novel compositions with improved effectiveness. The antimicrobial activity of these polymeric systems is closely associated with their three-dimensional structure, conformation, and available surface area. The development of novel active systems is in itself therefore not an adequate means of maximizing the utilization of full active potential.

This has also to be combined with optimization of the structure and of the available surface area. This applies particularly to coatings, molding compositions, semifinished products, and finished products in which a matrix of foreign molecules with no intrinsic antimicrobial activity surrounds the antimicrobial polymer.

It is therefore an object of the present invention to develop a process which renders surfaces antimicrobial, is simple to carry out, and has almost no dependency on the material of the surface to be treated.

The systems thus optimized are intended to be still more effective than the standard systems currently available in inhibiting the colonization and spread of bacteria, algae, and fungi on surfaces.

Surprisingly, it has now been found that through thermally assisted application of antimicrobial polymers it is possible to obtain surfaces which have high microbicidal activity.

It is not necessary here for antimicrobial polymers to be included in the materials incorporated into the substrate, for example during the compounding of molding compositions. Another advantage of the process is cost-effective reduction in the use of antimicrobial polymers, which in the event of compounding, for example, remain inactive within the matrix of the molding composition. Furthermore, this method can substantially avoid undesirable changes in the physical properties of the substrate, since only a very thin layer at the surface of the substrate undergoes modification. The process of the invention may readily be combined with other methods for the post-treatment of surfaces. For example, hydrophilicization with water or acids may be carried out after the thermally assisted application of the polymers. The surfaces thus treated have microbial activity which is longlasting and resistant to environmental effects and physical stresses. No low-molecular-weight biocides are present in these coatings, with the result that over the entire period of use there is effectively no possibility of migration of environmentally problematic substances.

The present invention therefore provides a process for rendering surfaces antimicrobial by thermal application of antimicrobial polymers to this surface.

To prepare the antimicrobial polymers it is preferable to use nitrogen- and phosphorus-functionalized monomers, and these polymers are in particular prepared from at least one of the following monomers: 2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methyacryloylaminopropyltrimethylammonium chloride, 2-methyacryloyloxyethyltrimethylammonium chloride, 2-acryloyloxyethyl-4-benzoyldiemthylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether.

One way of carrying out the process of the invention is to apply the antimicrobial polymers in finely divided form to the surface to be treated. The polymers are preferably in powder form when applied, in particular with a grain size of from 1 to 500 μm.

The surface is heated after or prior to the application of the antimicrobial polymer thereto.

The temperature of the surface here should at least reach the glass transition temperature of the antimicrobial polymer, and more preferably exceed that temperature. In the case of polymer surfaces, if the temperature moreover exceeds the glass transition temperature of these the resultant effect in combination with the antimicrobial polymer used can be formation of a microblend, still further improving the adhesion of the antimicrobial polymer on the surface. The polymer powder, which may, where appropriate, be finely milled and fractionated into defined grain sizes through screening prior to application, may be applied manually, e.g. by dusting by means of a sieve, or else ideally by a machine, e.g. through application by a blowing or dusting method directly downstream of an extruder. In the case of direct application after extrusion, i.e. application after heating of the surface, a fact which has proven to be a particular advantage is that reheating of the substrate to be coated can be avoided, thus avoiding additional costs and reducing the risk of damage to the substrate. In addition, the process can be inserted without difficulty within existing extrusion lines.

The surface to be rendered antimicrobial may be completely or partially covered by the polymers by means of the process of the invention.

Complete covering (i.e. a film) requires appropriate amounts of the polymer and, respectively, appropriate thermal treatment times. It is possible for the antimicrobial polymer merely to be subjected to incipient melting and/or for the amount applied to the surface to be so small that, where appropriate, the antimicrobial polymer retains a grain structure. The result is structuring of the surface.

In addition, the surface rendered antimicrobial may be hydrophilicized at or above the glass transition temperature of the antimicrobial polymer through contact with water or acids, in particular dilute organic or mineral acids. The increase in the concentration of hydrophilic groups, which are often a constituent of antimicrobial polymers, at the coated substrate interface still further promotes the antimicrobial action.

Use of Modified Polymer Substrates

The present invention also provides the use of the antimicrobial coatings optimized according to the invention for producing antimicrobial products, and the resultant products per se. These products are preferably based on polyamides, on polyurethanes, on polyether block amides, on polyester amides, or -imides, on PVC, on polyolefins, on silicones, on polysiloxanes, on polymethacrylate, or on polyterephthalates, on metals, on glasses, or on ceramics, which have surfaces coated with polymers of the invention.

Examples of particular antimicrobial products of this type are machine components for the processing of food or drink, air conditioning system components, coated pipes, semifinished products, roofing, bathroom or toilet items, kitchen items, components of sanitary equipment, components of animal cages—and housings, recreational products for children, components of water systems, packaging for food or drink, operating units (touch panels) of devices, and contact lenses.

The coatings of the invention may be used wherever importance is placed on surfaces which are as free as possible from bacteria, algae, and fungi, i.e. microbicidal surfaces or surfaces with release properties. Examples of the use of the coatings of the invention are found in the following sectors:

marine: ships' hulls, docks, buoys, drilling platforms, ballast water tanks

construction: roofing, basements, walls, facades, greenhouses, sun protection, garden fences, wood protection

sanitary: public conveniences, bathrooms, shower curtains, toilet items, swimming pools, saunas, jointing, sealing compounds

food and drink: machines, kitchens, kitchen items, sponges, recreational products for children, packaging for food or drink, milk processing, drinking water systems, cosmetics

machine parts: air conditioning systems, ion exchangers, processed water, solar-powered units, heat exchanges, bioreactors, membranes

medical technology: contact lenses, diapers, membranes, implants

consumer articles: automobile seats, clothing (socks, sports clothing), hospital equipment, door handles, telephone handsets, public conveyances, animal cages, cash registers, carpeting, wall-coverings

The present invention also provides items in medical technology or the use of the hygiene products produced according to the invention using processes or coatings optimized according to the invention. The above descriptions of preferred materials are again applicable. Examples of these hygiene products are toothbrushes, toilet seats, combs, and packaging materials. The term hygiene items also includes other articles which can sometimes come into contact with large numbers of people, examples being telephone handsets, stair rails, door handles, window catches, and grab straps and grab handles in public conveyances. Examples of items in medical technology are catheters, tubing, protective or backing films, and also surgical instruments.

The process of the invention may also be used to render pipes antimicrobial, e.g. in cooling water streams or in the agricultural or food technology sector (milk pipelines).

The examples below are given for further description of the present invention, and provide further illustration of the invention but are not intended to limit its scope as set out in the patent claims.

### EXAMPLE 1

50 ml of dimethylaminopropylmethacrylamide (Aldrich) and 250 ml of ethanol form an initial charge in a three-necked flask and are heated to 65° C. under a stream of argon. 0.6 g of azobisisobutyronitrile dissolved in 20 ml of ethyl methyl ketone is then slowly added dropwise, with stirring. The mixture is heated to 70° C. and stirred at this temperature for 72 hours. After expiry of this time, the reaction mixture is stirred into 1.5 of deionized water, whereupon the polymeric product precipitates. After isolation.of the product by filtration, the filter residue is washed with 100 ml of a mixture of ethanol and deionized water in a ratio of 1:1 in order to remove any residual monomers still present. The product is then dried in vacuo at 50° C. for 24 hours.

### EXAMPLE 1a

10 g of the polymer from example 1 are comminuted in a mortar. 0.5 g of the product comminuted in the mortar is then applied, through a sieve with mesh width 250 micrometers, to an aluminum plate of thickness 0.5 cm and dimensions 2×2 cm, which has been heated in advance to 110° C. The plate thus coated is then allowed to cool slowly to room temperature.

### EXAMPLE 1b

The coated aluminum plate from example 1a is placed with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 1c

The coated aluminum plate from example 1a is placed with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 1d

10 g of the polymer from example 1 are comminuted in a mortar. 0.5 g of the product comminuted in the mortar is then applied, through a sieve with mesh width 250 micrometers, to a VA plate of thickness 0.5 cm and dimensions 2×2 cm, which has been heated in advance to 110° C. The plate thus coated is then allowed to cool slowly to room temperature.

### EXAMPLE 1e

The coated VA plate from example 1d is placed with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 1f

The coated VA plate from example 1d is placed with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 1g

10 g of the polymer from example 1 are comminuted in a mortar. 0.5 g of the product comminuted in the mortar is then applied, through a sieve with mesh width 250 micrometers, to a PVC strip of thickness 0.1 cm and dimensions 4×8 cm, which has been heated in advance to 120° C. The PVC strip thus coated is then allowed to cool slowly to room temperature.

### Example 1h

The coated PVC strip from example 1 g is held with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 1i

The coated PVC strip from example 1 g is held with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 1j

The surface of the coated aluminum plate from example 1a is placed for 15 minutes in water heated to 60° C. The aluminum plate thus treated is then placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes in the test mixture is determined. After expiry of this time, the number of microbes has fallen from 10⁷ to 10⁴ microbes per ml.

### EXAMPLE 1k

The surface of the coated aluminum plate from example 1 a is placed for 15 minutes in water heated to 60° C. The aluminum plate thus treated is then placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of microbes in the test mixture is determined. After expiry of this time, no remaining microbes of Pseudomonas aeruginosa are detectable.

### EXAMPLE 2

50 ml of tert-butylaminoethyl methacrylate (Aldrich) and 250 ml of ethanol form an initial charge in a three-necked flask and are heated to 65° C. under a stream of argon. 0.6 g of azobisisobutyronitrile dissolved in 20 ml of ethyl methyl ketone is then slowly added dropwise, with stirring. The mixture is heated to 70° C. and stirred at this temperature for 72 hours. After expiry of this time, the reaction mixture is stirred into 1.5 l ,of deionized water, whereupon the polymeric product precipitates. After isolation of the product by filtration, the filter residue. is washed with 100 ml of a mixture of ethanol and deionized water in a ratio of 1:1 in order to remove any residual monomers still present. The product is then dried in vacuo at 50° C. for 24 hours.

### EXAMPLE 2a

10 g of the polymer from example 2 are comminuted in a mortar. 0.5 g of the product comminuted in the mortar is then applied, through a sieve with mesh width 250 micrometers, to an aluminum plate of thickness 0.5 cm and dimensions 2×2 cm, which has been heated in advance to 110° C. The plate thus coated is then allowed to cool slowly to room temperature.

### EXAMPLE 2b

The coated aluminum plate. from example 2a is placed with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 2c

The coated aluminum plate from example 2a is placed with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 2d

10 g of the polymer from example 2 are comminuted in a mortar. 0.5 g of the product comminuted in the mortar is then applied, through a. sieve with mesh width 250 micrometers, to a VA plate of thickness 0.5 cm and dimensions 2×2 cm, which has been heated in advance to 110° C. The plate thus coated is then allowed to cool slowly to room temperature.

### EXAMPLE 2e

The coated VA plate from example 2d is placed with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 2f

The coated VA plate from example 2d is placed with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 2g

10 g of the polymer from example 2 are comminuted in a mortar. 0.5 g of the product comminuted in the mortar is then applied, through a sieve with mesh width 250 micrometers, to a PVC strip of thickness 0.1 cm and dimensions 4×8 cm, which has been heated in advance to 120° C. The PVC strip thus coated is then allowed to cool slowly to room temperature.

### EXAMPLE 2h

The coated PVC strip from example 2 g is held with its coated side upward on the base of a glass beaker in which there are 20 ml of a test microbial suspension of Staphylococcus aureus, and is shaken. After a contact time of 4 hours, 1 ml of the test microbial suspension is removed, and the number. of microbes in the test mixture is determined. After expiry of this time, no remaining microbes of Staphylococcus aureus are detectable.

### EXAMPLE 2i

The coated PVC strip from example 2 g is held with its coated. side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 2j

The surface of the coated aluminum plate from example 2a is placed for 15 minutes in water heated to 60° C. The aluminum plate thus treated is then placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes in the test mixture is determined. After expiry of this time, the number of microbes has fallen from 10⁷ to 10⁴ microbes per ml.

### EXAMPLE 2k

The surface of the coated aluminum plate from example 2a is placed for 15 minutes in water heated to 60° C. The aluminum plate thus treated is then placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of microbes in the test mixture is determined. After expiry of this time, no remaining microbes of Pseudomonas aeruginosa are detectable.

### EXAMPLE 3

90 ml of 2-tert-butylaminoethyl methacrylate (Aldrich) and 180 ml of ethanol form an initial charge in a three-necked flask and are heated to 65° C. under a stream of argon. 0.745 g of azobisisobutyronitrile dissolved in 20 ml of ethyl methyl ketone is then slowly added dropwise, with stirring. The mixture is heated to 70° C. and stirred at this temperature for 72 hours. After expiry of this time, the reaction mixture is stirred into 1 l of demineralized water, whereupon the polymeric product precipitates. After isolation of the product by filtration, the filter residue is washed with 100 ml of a 10% strength solution of ethanol in water in order to remove any residual monomers still present. The product is then dried in vacuo at 50° C. for 24 hours.

### EXAMPLE 3a

68 g of polyvinyl chloride pellets and 32 g of diisononyl phthalate are mixed until intimate mixing has taken place and the mixture becomes a paste. 20 g of the resultant paste are spread, using a doctor, onto a metal plate so as to give a layer thickness of 0.7 mm. The plate with the paste thereon is then heated to 200° C. for 2 minutes, whereupon the paste gels and a plasticized PVC foil is produced.

### EXAMPLE 3b

10 g of the polymer from example 3 are comminuted in a mortar. 0.5 g of the product comminuted in the mortar is then applied, through a sieve with mesh width 250 micrometers, to a PVC foil from example 3a, which has been heated in advance. to 120° C. The PVC foil thus coated is then allowed to cool slowly to room temperature.

### EXAMPLE 3c

The coated PVC foil from example 3b is held with its coated side upward on the base of a glass beaker in which there are 20 ml of a test microbial suspension of Staphylococcus aureus, and is shaken. After a contact time of 4 hours, 1 ml of the test microbial suspension is removed, and the number of microbes in the test mixture is determined. After expiry of this time, no remaining microbes of Staphylococcus aureus are detectable.

### EXAMPLE 3d

The coated PVC foil from example 3b is held with its coated side upward on the base of a glass beaker in which there are 20 ml of a test microbial suspension of Pseudomonas aeruginosa, and is shaken. After a contact time of 4 hours, 1 ml of the test microbial suspension is removed, and the number of microbes in the test mixture is determined. After expiry of this time, no remaining microbes of Pseudomonas aeruginosa are detectable.

### EXAMPLE 4

90 ml of 2-tert-butylaminoethyl methacrylate (Aldrich) and 180 ml of ethanol form an initial charge in a three-necked flask and are heated to 65° C. under a stream of argon. 0.745 g of azobisisobutyronitrile dissolved in 20 ml of ethyl methyl ketone is then slowly added dropwise, with stirring. The mixture is heated to 70° C. and stirred at this temperature for 72 hours. After expiry of this time, the reaction mixture is stirred into 1 l of demineralized water, whereupon the polymeric product precipitates. After isolation of the product by filtration, the filter residue is washed with 100 ml of a 10% strength solution of ethanol in water in order to remove any residual monomers still present. The product is then dried in vacuo at 50° C. for 24 hours.

### EXAMPLE 4a

A brush is used to coat an acrylic lacquer from ROWA (Rowacryl G-31293) onto an aluminum plate of dimensions 5×5 cm, and the plate is then dried for a period of 24 hours at 35° C. in a drying cabinet. The coated aluminum plate is then heated to 110° C. 10 g of the polymer from example 4 are comminuted in a mortar. 0.5 g of the product comminuted in the mortar is then applied, through a sieve with a mesh width of 250 micrometers, to the heated aluminum plate. The plate thus coated is then allowed to cool slowly to room temperature.

### EXAMPLE 4b

This aluminum plate from example 4a is placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes in the test mixture is determined. After expiry of this time, no remaining microbes of Staphylococcus aureus are detectable.

### EXAMPLE 4c

This aluminum plate from example 4a is placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of microbes in the test mixture is determined. After expiry of this time, no remaining microbes of Pseudomonas aeruginosa are detectable.

### EXAMPLE 4d

A brush is used to coat an acrylic lacquer from ROWA (Rowacryl G-31293) onto a VA plate of dimensions 5×5 cm, and the plate is then dried for a period of 24 hours at 35° C. in a drying cabinet. The coated VA plate is then heated to 110° C. 10 g of the polymer from example 4 are comminuted in a mortar. 0.5 g of the product comminuted in the mortar is then applied, through a sieve with a mesh width of 250 micrometers, to the heated VA plate. The plate thus coated is then allowed to cool slowly to room temperature.

### EXAMPLE 4e

This VA plate from example 4d is placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes in the test mixture is determined. After expiry of this time, no remaining microbes of Staphylococcus aureus are detectable.

### EXAMPLE 4f

This VA plate from example 4d is placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of. microbes in the test mixture is determined. After expiry of this time, no remaining microbes of Pseudomonas aeruginosa are detectable.

### EXAMPLE 4g

A brush is used to coat an acrylic lacquer from ROWA (Rowacryl G-31293) onto a glass plate of dimensions 8×6 cm, and the plate is then dried for a period of 24 hours at 35° C. in a drying cabinet. The coated glass plate is then heated to 110° C. 10 g of the polymer from example 4 are comminuted in a mortar. 0.5 g of the product comminuted in the mortar is then applied, through a sieve with a mesh width of 250 micrometers, to the heated glass plate. The plate thus coated is then allowed to cool slowly to room temperature.

### EXAMPLE 4h

This glass plate from example 4g is placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes in the test mixture is determined. After expiry of this time, no remaining microbes of Staphylococcus aureus are detectable.

### EXAMPLE 4i

This glass plate from example 4 g is placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of microbes in the test mixture is determined. After expiry of this time, no remaining microbes of Pseudomonas aeruginosa are detectable.

### EXAMPLE 4j

A brush is used to coat an acrylic lacquer from ROWA (Rowacryl G-31293) onto a ceramic plate of dimensions 7×7 cm, and, the plate is then dried for a period of 24 hours at 35° C. in a drying cabinet. The coated ceramic plate is then heated to 110° C. 10 g of the polymer from example 4 are comminuted in a mortar. 0.5 g of the product comminuted in the mortar is then applied, through a sieve with a mesh width of 250 micrometers, to the heated aluminum plate. The plate thus coated is then allowed to cool slowly to room temperature.

### EXAMPLE 4k

This ceramic plate from example 4 j is placed with its coated side upward on the base of a glass beaker in which there are 20 ml of a test microbial suspension of Staphylococcus aureus, and is shaken. After a contact time of 2 hours, 1 ml of the test microbial suspension is removed, and the number of microbes. in the test mixture is determined. After expiry of this time, no remaining microbes of Staphylococcus aureus are detectable.

### EXAMPLE 4l

This ceramic plate from example 4 j is placed with its coated side upward on the base of a glass beaker in which there are 20 ml of a test microbial suspension of Pseudomonas aeruginosa, and is shaken. After a contact time of 2 hours, 1 ml of the test microbial suspension is removed, and the number of microbes in the test mixture is determined. After expiry of this time, no remaining microbes of Pseudomonas aeruginosa are detectable.

What is claimed is:

1. A process for rendering surfaces antimicrobial by thermal application of antimicrobial polymers to this surface.

2. The process as claimed in claim 1, characterized in that prior to the application of the antimicrobial polymer, the surface to be rendered antimicrobial is heated at least to the glass transition temperature of the antimicrobial polymer.

3. The process as claimed in claim 1, characterized in that after application of the antimicrobial polymer, the surface to be rendered antimicrobial is heated at least to the glass transition temperature of the antimicrobial polymer.

4. The process as claimed in any of claims 1 to 3, characterized in that the antimicrobial polymer is a powder with grain size of from 1 to 500 μm when applied to the surface to be rendered antimicrobial.

5. The process as claimed in any of claims 1 to 4, characterized in that the surface to be rendered antimicrobial is completely covered with antimicrobial polymers as a result of the thermal application.

6. The process as claimed in any of claims 1 to 5, characterized in that the surface to be rendered antimicrobial is partially covered with antimicrobial polymers as a result of the thermal application.

7. The process as claimed in any of claims 1 to 6, characterized in that the surfaces to be rendered antimicrobial are composed of polymers, of ceramics, of glasses, of metals, or of wood.

8. The process as claimed in any of claims 1 to 7, characterized in that the surfaces to be rendered antimicrobial are hydrophilicized at or above the glass transition temperature of the antimicrobial polymer, using acids or water.

9. The process as claimed in any of claims 1 to 8, characterized in that the antimicrobial polymers are prepared from at least one nitrogen- or phosphorus-functionalized monomer.

10. The process as claimed in any of claims 1 to 8, characterized in that the antimicrobial polymers have been prepared from at least one of the following monomers: 2-tert-butylaminoethyl methacrylate, 2-diethyl-aminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methyacryloylaminopropyltrimethylammonium chloride, 2-methyacryloyloxyethyltrimethylammonium chloride, 2-acryloyloxyethyl-4-benzoyldiemthylammonium bromide, 2-methacryloyloxyethyl-4-benzoyldimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether.
